(19) United States
(12) Patent Application Publication
(10) Pub. No.: US 2025/0288529 A1
(43) Pub. Date: Sep. 18, 2025

(54) PROTEIN ENCAPSULATION OF NUTRITIONAL AND PHARMACEUTICAL COMPOSITIONS

(71) Applicant: Clover Corporation Limited, Altona North (AU)

(72) Inventors: Lourdes URBAN-ALANDETE, Camp Hill (AU); Glenn ELLIOTT, Casuarina (AU); Mek Cheng, Calmvale (AU); Bo WANG, Parklea (AU); Jessica RYAN, Coorparoo (AU)

(73) Assignee: Clover Corporation Limited, Altona North (AU)

(21) Appl. No.: 19/098,877

(22) Filed: Apr. 2, 2025

Related U.S. Application Data
(63) Continuation of application No. 17/261,323, filed on Jan. 19, 2021, now abandoned.

(30) Foreign Application Priority Data
Jul. 24, 2018 (AU) 2018902668

(51) Int. Cl. A61K 9/50; A23D 7/005; A23D 7/01; A23D 9/05; A23L 33/12; B01J 13/04

(52) U.S. Cl. CPC A61K 9/5015; A23D 7/0053; A23D 7/011; A23D 9/05; A23L 33/12; A61K 9/5036; A61K 9/5052; B01J 13/043

(57) ABSTRACT

Provided herein are microencapsulated compositions, optionally oil-in-water emulsions, comprising one or more long chain polyunsaturated fatty acids (LCPUFAs), optionally in triglyceride form, wherein the encapsulant comprises one or more low molecular weight proteins. In particular embodiments the composition has a surface free fat content of about 1%. Also provided are methods for protecting one or more LCPUFAs, or one or more oils comprising the one or more LCPUFAs, from oxidative degradation, comprising encapsulating the LCPUFAs or oil with an encapsulant comprising one or more low molecular weight proteins.

### PRIORITY CLAIM TO RELATED APPLICATIONS

This application is a continuation of U.S. Ser. No. 17/261,323, filed on Jan. 19, 2021, which is a U.S. national stage filing under 35 U.S.C. § 371 from International Application No. PCT/AU2019/050763, filed on Jul. 23, 2019, and published as WO 2020/019019 on Jan. 30, 2020, which claims the benefit under 35 U.S.C. 119 to Australian Application No. 2018902668, filed on Jul. 24, 2018, the benefit of priority of each of which is claimed herein, and which applications and publication are hereby incorporated herein by reference in their entirety.

### TECHNICAL FIELD

The present disclosure broadly relates to encapsulated compositions comprising long-chain polyunsaturated fatty acid (LCPUFA)-containing oils suitable for both nutritional and pharmaceutical applications and to means for protecting LCPUFA-containing oils in encapsulated compositions from oxidation and oxidative degradation.

### BACKGROUND OF THE DISCLOSURE

It is well known that long-chain polyunsaturated fatty acids (LCPUFAs) are an important nutritional component of the human diet and that many people fail to consume an adequate amount of these essential fatty acids, in particular omega-3 fatty acids such as eicosapentaenoic acid (EPA) and docosahexaenoic acid (DHA). A large number of studies have found that omega-3 fatty acids play an influential role in heart, brain and eye health. For example, a recent study suggests that EPA and DHA may have the ability to decrease heart rate and oxygen consumption during exercise, therefore contributing to enhanced physical and mental performance in athletes (People et al., Journal of Cardiovascular Pharmacology, 2008, 52:540-547). Because of their essential nutritional role, compositions comprising omega-3 fatty acids are important in terms of both nutritional supplementation, and as pharmaceutical agents.

Accordingly, there is a growing trend towards incorporating omega-3 fatty acids, for example, fish oils, algal oils and some plant seeds oils, into food products to promote public health. However, due to the susceptibility of these fatty acids to oxidation or degradation upon exposure to oxygen, elevated temperature or light, which are common occurrences during food production and storage, it is a challenge to successfully fortify products with omega-3 fatty acids, maintaining stability and activity of the omega-3 fatty acids. The oxidation and/or degradation of omega-3 fatty acids generates undesirable oxidation breakdown products which may adversely affect the organoleptic properties or physiological properties of the formulation.

Microencapsulation technology, through which bioactive compounds can be entrapped within physical protective shell materials, has been successfully used to protect omega-3 fatty acids against oxidation and degradation. Spray drying is the most widely used technique to produce microcapsule powders. Typically, spray dried microcapsule powders containing omega-3 rich oils and have an oil loading of approximately 30% (w/w) and a surface free fat content of approximately 1% (w/w). Due to the superior functional properties of Maillard reaction products (MRPs), omega-3 oil-containing microcapsule powders have been produced with an oil loading as high as 48±2%, while maintaining a surface free fat content of approximately 1% (w/w).

Notwithstanding, there is a need for the development of encapsulation and delivery systems capable of improving the oxidative stability of omega-3 rich oils.

### SUMMARY OF THE DISCLOSURE

The present disclosure is predicated on the inventors' unexpected discovery that the oxidative stability of compositions and emulsions comprising omega-3 rich oils can be improved by the use of an encapsulant comprising one or more low molecular weight proteins or an emulsifier comprising low molecular weight proteins fractions.

A first aspect of the present disclosure provides a microencapsulated composition comprising one or more long chain polyunsaturated fatty acids (LCPUFAs), wherein the encapsulant comprises one or more low molecular weight proteins.

In an embodiment the molecular weight of the one or more proteins is less than about 50 kDa, less than about 40 kDa, less than about 30 kDa, or less than about 20 kDa. The proteins may be in the form of a protein fraction, optionally from a natural source. In an exemplary embodiment the proteins are in the form of a potato protein fraction comprising proteins with molecular weights less than about 30 kDa, or less than about 20 kDa.

In an embodiment, the encapsulant further comprises one or more carbohydrates. In an exemplary embodiment the one or more carbohydrates are selected from maltodextrin and dextrose monohydrate. In an exemplary embodiment the carbohydrates comprise maltodextrin and dextrose monohydrate.

In particular embodiments, the ratio (by weight) of the protein component of the encapsulant to the carbohydrate component of the encapsulant may be from about 1:10 to about 1:1.5. In exemplary embodiments the protein component of the encapsulant comprises from about 15% w/w to about 21% w/w based on the total weight of the composition. In exemplary embodiments the ratio (by weight) of the protein component of the encapsulant to the carbohydrate component of the encapsulant is about 1:2.

In an embodiment, the LCPUFAs are omega-3 fatty acids, such as DHA and/or EPA. The LCPUFAs may be present, for example, in triglyceride form or in phospholipid form. In particular embodiments as disclosed herein the LCPUFAs are present in triglyceride form. The LCPUFAs may be present in one or more LCPUFA-containing oils. The LCPUFA-containing oil(s) may be naturally occurring or naturally derived, or may be synthetic. The LCPUFA-containing oil(s) may be rich in LCPUFAs. In an exemplary embodiment the oil is a fish oil, such as tuna oil.

In an embodiment, the composition further comprises a vitamin C source. In an exemplary embodiment the vitamin C source is ascorbic acid or sodium ascorbate.

Typically the composition has a surface free fat content of less than about 2%. In an embodiment, the composition has a surface free fat content of about 1%.

The composition may be in the form of an emulsion, such as on oil-in-water emulsion. The composition may be in the form of a powder, such as a spray dried powder. The powder may be obtained by drying an oil-in-water emulsion.

A second aspect of the present disclosure provides a method for protecting one or more LCPUFAs from oxidative degradation, comprising encapsulating an oil comprising the one or more LCPUFAs with an encapsulant comprising one or more low molecular weight proteins.

In an embodiment the molecular weight of the one or more proteins is less than about 50 kDa, less than about 40 kDa, less than about 30 kDa, or less than about 20 kDa. The proteins may be in the form a of a protein fraction, optionally from a natural source. In an exemplary embodiment the proteins are in the form of a potato protein fraction comprising proteins with molecular weights less than about 30 kDa, or less than about 20 kDa.

In an embodiment, the encapsulant further comprises one or more carbohydrates. In an exemplary embodiment the one or more carbohydrates are selected from maltodextrin and dextrose monohydrate. In an exemplary embodiment the carbohydrates comprise maltodextrin and dextrose monohydrate.

In particular embodiments, the ratio (by weight) of the protein component of the encapsulant to the carbohydrate component of the encapsulant may be from about 1:10 to about 1:1.5. In exemplary embodiments the protein component of the encapsulant comprises from about 15% w/w to about 21% w/w based on the total weight of the composition. In exemplary embodiments the ratio (by weight) of the protein component of the encapsulant to the carbohydrate component of the encapsulant is about 1:2.

In an embodiment, the LCPUFAs are omega-3 fatty acids, such as DHA and/or EPA. The LCPUFAs may be present, for example, in triglyceride form or in phospholipid form. In particular embodiments as disclosed herein the LCPUFAs are present in triglyceride form. The LCPUFAs may be present in one or more LCPUFA-containing oils. The LCPUFA-containing oil(s) may be naturally occurring or naturally derived, or may be synthetic. The LCPUFA-containing oil(s) may be rich in LCPUFAs. In an exemplary embodiment the oil is a fish oil, such as tuna oil.

In an embodiment, the composition further comprises a vitamin C source. In an exemplary embodiment the vitamin C source is ascorbic acid or sodium ascorbate.

Typically the composition has a surface free fat content of less than about 2%. In an embodiment, the composition has a surface free fat content of about 1%.

The composition may be in the form of an emulsion, such as on oil-in-water emulsion. The composition may be in the form of a powder, such as a spray dried powder. The powder may be obtained by drying an oil-in-water emulsion.

In a third aspect of the present disclosure there is provided a method for improving the oxidative stability of one or more LCPUFAs from oxidative degradation, comprising encapsulating an oil comprising the one or more LCPUFAs with an encapsulant comprising one or more low molecular weight proteins.

A fourth aspect of the present disclosure provides a stable emulsion comprising one or more LCPUFAs, optionally an oil comprising the one or more LCPUFAs, wherein said emulsion further comprises one or more low molecular weight proteins.

Typically the emulsion is an oil-in-water emulsion.

A fifth aspect of the present disclosure provides a composition comprising one or more LCPUFAs, optionally an oil comprising the one or more LCPUFAs, and one or more low molecular weight proteins.

The composition may be in the form of an emulsion, such as on oil-in-water emulsion. The composition may be in the form of a powder, such as a spray dried powder.

### BRIEF DESCRIPTION OF THE FIGURES

Exemplary embodiments of the present disclosure are described herein, by way of non-limiting example only, with reference to the following drawings.

FIG. 1. The molecular weight (kDa) of proteins/protein fractions: M, molecular weight markers; PPH, potato protein faction with high molecular weight (two lanes); PPL, potato protein faction with low molecular weight (two lanes); SC, sodium caseinate; MRPs, Maillard reaction products between SC and carbohydrate polymers; WPI, whey protein isolate.

FIG. 2. Exemplary process flow of microencapsulation of LCPUFA-rich tuna oil with proteins (PPH, PPL, SC and WPI), dextrose monohydrate (DM) and maltodextrin (MAL) or MRPs as encapsulants, as described in Example 1.

FIG. 3. Induction period of various tuna oil (TO)-containing microcapsule powders in different microencapsulation matrices at 70° C. and 5 bar oxygen. From left to right in the graph (based on location of the arrowed circle): MRPs-based TO powder ˜50% oil loading, Maillard reaction products between SC and carbohydrate polymers as encapsulants; WPI-based TO powder ˜30% oil loading, whey protein isolate and carbohydrate polymers as encapsulants; PPL-based TO powder ˜50% oil loading, potato protein faction with low molecular weight and carbohydrate polymers as encapsulants; and SC-based TO powder ˜30% oil loading, sodium caseinate and carbohydrate polymers as encapsulants.

### DETAILED DESCRIPTION

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” is understood to refer to a range of numbers that a person of skill in the art would consider equivalent to the recited value in the context of achieving the same function or result.

In the context of this specification, the terms “a” and “an” refer to one or to more than one (i.e. to at least one) of the grammatical object of the article. By way of example, “an element” means one element or more than one element.

As used herein, the term “oxidative stability” in relation to LCPUFAs, means the stability of the LCPUFAs or a LCPUFA-containing oil in the presence of oxygen and resistance to oxidation or oxidative degradation. Thus, a higher oxidative stability is indicative of greater resistance to oxidation and oxidative degradation. Typically reference to improved oxidative stability resulting from encapsulation in accordance with the present disclosure means an improvement over the oxidative stability observed in the absence of an encapsulant according to the present disclosure or in the presence of an alternative encapsulant.

Particular embodiments of the present disclosure provide microencapsulated compositions comprising one or more long chain polyunsaturated fatty acids (LCPUFAs), wherein the encapsulant comprises one or more low molecular weight proteins.

Also provided are methods and compositions in which one or more low molecular weight proteins are used to encapsulate one or more LCPUFAs or an oil comprising the one or more LCPUFAs, to protect the LCPUFAs or LCPUFA-containing oil from oxidation or oxidative degradation. The protection from oxidation or oxidative degradation may be determined by any suitable means well known to those skilled in the art.

Microencapsulated compositions of the present disclosure may be in the form of, for example, an emulsion or may be in a solid form. The emulsion may comprise an oil-in-water emulsion. The solid form may be a powder. The powder may be obtained by spray drying, for example of an emulsion. In one embodiment, the composition is a free-flowing powder. The powder may have a mean particle size between about 10 □m and 1000 □m, or between about 50 □m and 800 □m, or between about 100 □m and 300 □m. In alternative embodiments the composition may be in the form of granules.

Compositions of the present disclosure are generated by microencapsulation, wherein the encapsulant comprises or consists of one or more low molecular weight proteins. The one or more proteins may be isolated proteins or may be in the form of a protein fraction obtained, for example, from a natural source, such as a cellular or tissue source. The cellular or tissue source may be obtained from any suitable source, such as an animal or plant source. The molecular weight of the proteins may be, for example, in the range of about 1 kDa to about 50 kDa, about 4 kDa to about 40 kDa or about 10 kDa to about 30 kDa. For example, the proteins may have a molecular weight of up to about 1 kDa, 2 kDa, 4 kDa, 6 kDa, 8 kDa, 10 kDa, 12 kDa, 14 kDa, 16 kDa, 18 kDa, 20 kDa, 22 kDa, 24 kDa, 26 kDa, 28 kDa, 30 kDa, 32 kDa, 34 kDa, 36 kDa, 38 kDa, 40 kDa, 42 kDa, 44 kDa, 46 kDa, 48 kDa or up to about 50 kDa. In the case of a protein fraction, it is not essential that proteins present in the fraction have a molecular weight in the ranges defined above, but that at least one of the proteins in the fraction has a molecular weight falling within the above ranges.

The scope of the present disclosure should not be limited by reference to any specific proteins. In an exemplary embodiment, the low molecular weight proteins are in the form of a potato protein fraction that may comprise protease inhibitors such as protease inhibitor I (about 39 kDa), carboxypeptidase inhibitor (about 4100 Da), protease inhibitor IIa and IIb (about 20.7 kDa) and protease inhibitor A5 (about 20.7 kDa).

The one or more low molecular weight proteins may be introduced into the emulsion or composition at any stage in the preparation of the emulsion or composition such that a homogenous aqueous dispersion or slurry is formed. Those skilled in the art will be able to optimise the amount and molecular weights of the proteins to be introduced without undue burden or experimentation. The molecular weight of the protein(s) should be sufficiently low to facilitate microencapsulation while the amount of said protein(s) should be sufficient to provide effective protection as the encapsulant. In the case of oil-in-water emulsions, the viscosity should also be controlled. If the viscosity is too high spray drying may be hindered. Determining the appropriate protein content and the appropriate viscosity is well within the capabilities of the skilled person.

In exemplary, the one or more low molecular weight proteins may be present at between about 5% (w/w) and about 30% (w/w) based on the total weight of the composition. In the case of an oil-in-water emulsion, this means between about 5% (w/w) and about 30% (w/w) based on the total weight of the aqueous phase plus the oil phase. For example, the one or more proteins may be present at about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% w/w based on the total weight of the composition.

In particular embodiments described herein the encapsulant comprises compounds, substances or moieties in addition to the one or more low molecular weight proteins. For example, the encapsulant may comprise a combination of one or more low molecular weight proteins with one or more polysaccharide or carbohydrate components. For example, a carbohydrate with a reducing sugar functional group may be reacted with the protein. dextrose (including dextrose monohydrate), glucose, lactose, sucrose, oligosaccharide and dried glucose syrup. In a further embodiment a polysaccharide, high-methoxy pectin or carrageenan, may be added to protein-carbohydrate mixtures in some formulations. Care needs to be taken in reacting the protein and carbohydrate to ensure that the conditions do not result in extensive gelling or coagulation of the protein, as this will render the protein incapable of forming a good film.

In an exemplary, compositions of the present disclosure may be prepared by solubilising the protein and the polysaccharide or carbohydrate components of the encapsulant in an aqueous phase, optionally using a high shear mixer. The mixture may then be heated to a temperature of about 60° C. to 80° C. after which time one or more antioxidants may be added if desired. The LCPUFA-containing oil may be dosed in-line to the aqueous mixture which is passed through a high shear mixer to form a coarse emulsion. The coarse emulsion may then be passed through homogenisation. If it is desired to prepare a powdered product the emulsion may be pressurised and spray-dried at an inlet temperature of about 180° C. and an outlet temperature of 80° C.

By way of example, a suitable polysaccharide and carbohydrate component may comprise maltodextrin, dextrose (including dextrose monohydrate), glucose, lactose, sucrose, oligosaccharide and dried glucose syrup, or combinations of one or more thereof. In a further embodiment a polysaccharide, high-methoxy pectin or carrageenan, may be added to protein-carbohydrate mixtures in some formulations. Care needs to be taken in reacting the protein and carbohydrate to ensure that the conditions do not result in extensive gelling or coagulation of the protein, as this will render the protein incapable of forming a good film. The ratio (by weight) of the low molecular weight proteins to the polysaccharide or carbohydrate component of the encapsulant may be, for example, about 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10.

In particular embodiments, the ratio (by weight) of the protein component of the encapsulant to the carbohydrate component of the encapsulant may be from about 1:10 to about 1:1.5. For example, the ratio of protein component to carbohydrate component may be about 1:10, 1:9.5, 1:9, 1:8.5, 1:8, 1:7.5, 1:7, 1:6.5, 1:6, 1:5.5, 1:5, 1:4.5, 1:4, 1:3.5, 1:3, 1:2.5, 1:2 or 1:1.5. The ratio of protein component to carbohydrate component may be from about 1:3 to about 1:1.5, for example about 1:3, 1:2.9, 1:2.8, 1:2.7, 1:2.6, 1:2.5, 1:2.4, 1:2.3, 1:2.2, 1:2.1, 1:2, 1:1.9, 1:1.8, 1:1.7, 1:1.6 or 1:1.5. The ratio of protein component to carbohydrate component may be from about 1:2 to about 1:1.9, for example about 1:2, 1:1.99, 1:1.98, 1:1.97, 1:1.96, 1:1.95, 1:1.94, 1:1.93, 1:1.92, 1:1.91 or 1:1.9. In an exemplary embodiment, the ratio of protein component to carbohydrate component is about 1:1.94.

The polysaccharide or carbohydrate component may have a DE value of between about 0 and 100, about 10 and 70, about 20 and 60, or about 30 and 50. The DE value may be about 0, 5, 10, 15, 20, 25, 30, 35, 40, 45, 50, 55, 60, 65, 70, 75, 80, 85, 90, 95 or 100.

The skilled addressee will appreciate that alternative carbohydrate sources may also be employed in the encapsulant in combination with the one or more low molecular weight proteins. For example, the carbohydrate source may comprise octenylsuccinic anhydride-modified starch and one or more, or two or more, sources of reducing sugars, with dextrose equivalent values of between about 0 and 80 as has been described previously in WO2012/106777, the disclosure of which is incorporated herein by reference. Briefly, the starch may comprise primary and/or secondary modifications and may be an ester or half ester. Suitable octenylsuccinic anhydride-modified starches include, for example, those based on waxy maize and sold under the trade names PURITY GUM®, CAPSUL® IMF and HI CAP® IMF by Ingredion ANZ Pty Ltd, Seven Hills, NSW, Australia. The octenylsuccinic anhydride-modified starch may be present in an amount of less than about 18%, 17%, 16%, 15%, 14%, 13%, 12%, 11%, 10%, 9%, 8%, 7%, 6.5%, 6%, 5.5%, 5%, 4.5%, 4%, 3.5%, 3%, 2.5%, 2% or less than 1%, of the total weight of the composition.

Sources of reducing sugars are well known to those skilled in the art and include monosaccharides and disaccharides, for example glucose, fructose, maltose, galactose, glyceraldehyde and lactose. Suitable sources of reducing sugars also include oligosaccharides, for example glucose polymers, such as dextrin and maltodextrin and glucose syrup solids. The reducing sugars may also be derived from glucose syrup which typically contains not less than 20% by weight of reducing sugars.

The surface free fat content of a microencapsulated composition according to the present disclosure may be less than or about 10%, less than or about 9%, less than or about 8%, less than or about 7%, less than or about 6%, less than or about 5%, less than or about 4%, less than or about 3%, less than or about 2.5%, less than or about 2.4%, less than or about 2.3%, less than or about 2.2%, less than or about 2.1%, less than or about 2%, less than or about 1.9%, less than or about 1.8%, less than or about 1.7%, less than or about 1.6%, less than or about 1.5%, less than or about 1.4%, less than or about 1.3%, less than or about 1.2%, less than or about 1.1%, or less than or about 1%. In particular embodiments, this surface free fat content is determined in a powder derived or produced from an emulsion.

Compositions and emulsions of the present disclosure comprise one or more LCPUFAs or an oil(s) comprising the one or more LCPUFAs. The oil(s) may be naturally occurring or naturally derived, or may be synthetic from genetically modified or non-genetically modified source. In the contest of the present disclosure “naturally occurring” and “naturally derived” includes oils and lipid compositions that may be extracted from a natural source such as the organisms listed herein, or that may be derived from or modified from an oil or one or more lipids found in such natural sources. The skilled person will appreciate that scope of the present disclosure is not limited by reference to the identity or source of the one or more LCPUFAs or oil(s) comprising the one or more LCPUFAs.

Exemplary oils that are, or can be modified to be LCPUFA-containing or LCPUFA-rich, include oils from marine organisms such as, for example, crustaceans such as krill, molluscs such as oysters, and fish such as tuna, salmon, trout, sardines, mackerel, sea bass, menhaden, herring, pilchards, kipper, eel or whitebait. The oil may be from the roe of one or marine organisms such as those listed herein. In exemplary embodiments, the oil is or comprises tuna oil, krill oil or a lipid extract from fish roe.

Other exemplary oils that are, or may be modified to be LCPUFA-containing or LCPUFA-rich, include plant sources and microbial sources. Plant sources include, but are not limited to, flaxseed, walnuts, sunflower seeds, canola, safflower, soy, wheat germ, corn and leafy green plants such as kale, spinach and parsley. Microbial sources include algae and fungi.

The oil(s) may be present in an amount between about 0.1% and 80% of the total weight of the composition, or in an amount between about 1% and 80%, or in an amount between about 1% and 75%, or in an amount between about 5% and 80%, or in an amount between about 5% and 75%, or in an amount between about 5% and 70% of the total weight of the composition. In exemplary embodiments, where the oil is tuna oil, the oil may be present in an amount of about 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78% or 80% of the total weight of the composition.

The LCPUFAs typically comprise one or more omega-3 fatty acids and/or one or more omega-6 fatty acids, or mixtures thereof. The fatty acids may include DHA, AA, EPA, DPA and/or stearidonic acid (SDA), or mixtures thereof. In one embodiment, the fatty acids comprise DHA and EPA.

Compositions contemplated by the present disclosure may further comprise additional components, for example, antioxidants, anti-caking agents, flavouring agents, colouring agents, vitamins, minerals, amino acids, chelating agents and the like.

Suitable antioxidants are well known to those skilled in the art, and may be water soluble or oil soluble. Suitable water soluble antioxidants include, for example, sodium ascorbate, calcium ascorbate, potassium ascorbate, ascorbic acid, glutathione, lipoic acid and uric acid. In an embodiment the water soluble antioxidant may be present in the composition in a range of about 0-10% wt/wt of the total composition. Suitable oil soluble antioxidants include, for example, tocopherols, ascorbyl palmitate, tocotrienols, phenols, polyphenols and the like. In an embodiment the oil soluble antioxidant is present in the oil phase in a range of about 0-10% wt/wt of the total composition.

Anti-caking agents that are compatible with the compositions of the present disclosure will be well known amongst those skilled in the art and include calcium phosphates, such as tricalcium phosphate and carbonates, such as calcium and magnesium carbonate and silicon dioxide

The compositions may further comprise one or more low molecular weight emulsifiers. Suitable low molecular weight emulsifiers include, for example, mono-and di-glycerides, lecithin and sorbitan esters. Other suitable low molecular weight emulsifiers will be well known to those skilled in the art. The low molecular weight emulsifier may be present in an amount between about 0.1% and 3% of the total weight of the composition, or in an amount between about 0.1% and about 2%, or in an amount between about 0.1% and 0.5%, or in an amount between about 0.1% and 0.3%, of the total weight of the composition. For example, the low molecular weight emulsifier may be present in an amount of about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2% of the total weight of the composition.

Compositions contemplated herein may be formulated for administration to subjects by any suitable route, typically oral administration. The composition may be in liquid or solid form, and may be consumed as such (for example in the form of a syrup or other suitable liquid, or as capsules or other suitable solid form). Alternatively, the compositions may be incorporated into food or beverage products.

It will be appreciated by persons skilled in the art that numerous variations and/or modifications may be made to the invention without departing from the spirit or scope of the invention as broadly described. The present embodiments are, therefore, to be considered in all respects as illustrative and not restrictive.

The present invention will now be further described in greater detail by reference to the following specific examples, which should not be construed as in any way limiting the scope of the invention.

### Examples

### Example 1—Encapsulation of Phospholipid-Containing Oils Using Low Molecular Weight Protein Encapsulants

Various proteins and protein fractions of differing molecular weight were investigated for their ability to stabilise microencapsulated phospholipid-rich oils in the form of spray dried powders. A refined tuna oil containing mixed natural tocopherols and ascorbyl palmitate was used as the core material. Several proteins and protein fractions with varying molecular weights, including sodium caseinate, whey protein isolate, high molecular weight potato protein fraction, and low molecular weight potato protein fraction were selected as the protein source for encapsulation.

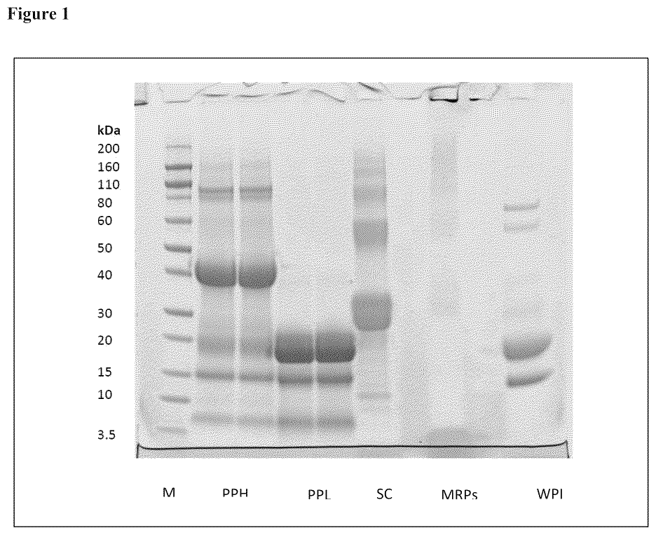

The molecular weight of these proteins and protein fractions were determined using SDS-PAGE under non-reducing conditions. The results are shown in FIG. 1. Sodium caseinate exhibits a band at approximately 30 kDa for casein proteins, approximately 14 kDa as α-lactoglobulin and a smear above 50 kDa, which may represent κ-casein and αₛ₂-casein. Resulting from the Maillard reaction, sodium caseinate was bound with carbohydrate polymers, and thus its molecular weight significantly increased; the molecular weight of Maillard reaction products (MRPs) exceeded 200 kDa. Whey protein isolate (WPI) showed bands at 14 and 18 kDa for α- and β-lactoglobulin, 66 kDa for bovine serum albumin and a band between 160 and 200 kDa. The high molecular weight potato protein fraction (PPH) exhibited a major band at approximately 40 kDa for patatin, together with the dimer at approximately 80 kDa. In the low molecular weight potato protein fraction (PPL), bands for protease inhibitors below 40 kDa were detected, suggesting the presence of protease inhibitor I (molecular weight 39 kDa), carboxypeptidase inhibitor (molecular weight 4100 Da), protease inhibitor IIa and IIb (molecular weights of about 20.7 kDa) and protease inhibitor A5 (molecular weight about 20.7 kDa).

Tuna oil was microencapsulated using proteins and protein fractions as described above as protein source, together with carbohydrate polymers dextrose monohydrate and maltodextrin (DE values of approximately 30), if required. Sodium ascorbic and ascorbic acid were used as vitamin C source in neutral and low pH conditions, respectively.

In order to eliminate the effect of varying surface free fat on the oxidative stability of the final spray dried powders, microcapsules with similar surface free fat content (approximately 1%) were produced. For this purpose, the oil loading of sodium caseinate-and WPI-based microcapsules was controlled at 28±2% (see Table 1) while the oil loading of MRP-, PPH- and PPL-based microcapsules was controlled at 48±2% (see Table 2).

TABLE 1
Sodium caseinate and WPI-based microcapsule
powders with 28 ± 2% tuna oil loading

| SC-based microcapsules | | WPI- based microcapsules | |
|---|---|---|---|
| Ingredients | Percentage (%) | Ingredients | Percentage (%) |
| Aqueous phase | | Aqueous phase | |
| SC | 21.85 | WPI | 21.85 |
| DM | 20.70 | DM | 20.70 |
| MAL | 21.85 | MAL | 21.85 |
| SA | 5.35 | SA | 5.35 |
| Oil phase | | Oil phase | |
| TO | 30 | TO | 30 |
| Total oil | 28.6 | Total oil | 28.6 |
| loading (~%) | | loading (~%) | |

SC, sodium caseinate; WPI, whey protein isolate; DM, dextrose monohydrate; MAL, maltodextrin (with DE value of approximately 30); SA, sodium ascorbate; TO, tuna oil.

TABLE 2
MRP-, PPH- and PPL-based microcapsule powders with 48 ± 2% tuna oil loading

| MRPs-based microcapsules | | PPH- based microcapsules | | PPL- based microcapsules | |
|---|---|---|---|---|---|
| | Percentage | | Percentage | | Percentage |
| Ingredients | (%) | Ingredients | (%) | Ingredients | (%) |
| Aqueous phase | | Aqueous phase | | Aqueous phase | |
| MRPs | | | | | |
| SC | 15.3 | PPH | 15.3 | PPL | 15.45 |
| DM | 14.49 | DM | 14.49 | DM | 14.64 |
| MAL | 15.3 | MAL | 15.3 | MAL | 15.45 |
| SA | 5.35 | SA | 5.35 | AA | 4.44 |
| Oil phase | | Oil phase | | Oil phase | |
| TO | 50 | TO | 50 | TO | 50 |
| Total oil | 47.8 | Total oil | 47.8 | Total oil | 48.3 |
| loading (~%) | | loading (~%) | | loading (~%) | |

MRPs, Maillard reaction products between sodium caseinate and carbohydrate polymers; PPH, high molecular weight potato protein fraction; PPL, low molecular weight potato protein fraction; SC, sodium caseinate; DM, dextrose monohydrate; MAL, maltodextrin (with DE value of approximately 30); SA, sodium ascorbate; AA, ascorbic acid; TO, tuna oil.

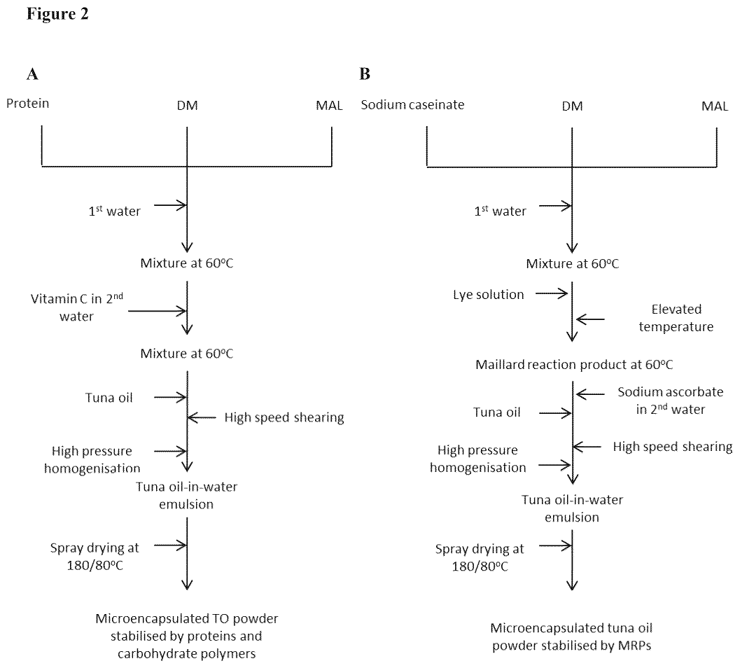

The microencapsulation process flow is shown in FIG. 2. As shown in FIG. 2A, for microcapsules generated using SC, WPI, PPH and PPL (collectively ‘protein’ in FIG. 2A), the encapsulant ingredients were added to water at 60° C. and subsequently heated to 80° C. After sodium ascorbate or ascorbic acid was added to the aqueous phase, refined tuna oil was homogenised in the mixture using a high-shear mixer to produce a coarse oil-in-water emulsion. This emulsion was further homogenised using a 2-stage homogeniser at 600 bar for 3 passes, followed by spray drying at 180/80° C.

As shown in FIG. 2B for microcapsules generated using MRPs, the Maillard reaction was induced prior to encapsulation. Briefly, sodium caseinate, dextrose monohydrate and maltodextrin were added to water at 60° C. and the pH was adjusted to 7-7.5.The slurry was heated at 90° C. to induce the Maillard reaction and the produced MRPs were cooled down to 80° C. After adding sodium ascorbate refined tuna oil was homogenised in the aqueous phase using a high-shear mixer, followed by homogenisation using a 2-stage homogeniser at 600 bar for 3 passes followed by spray drying at 180/80° C.

### Example 2—Oxidative Stability of Microencapsulated Powders of Example 1

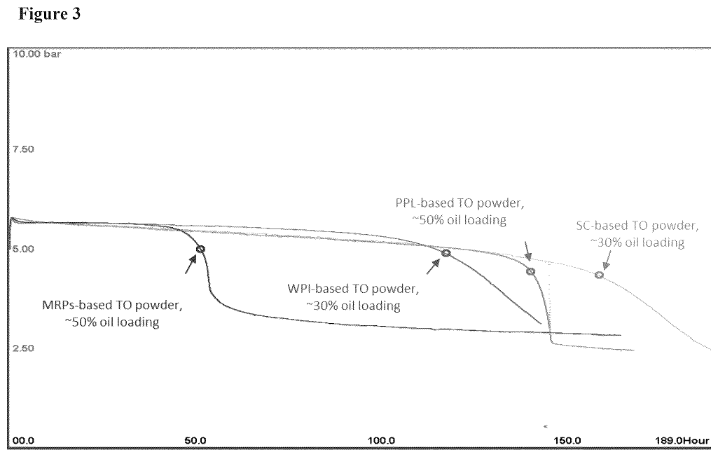

Microencapsulated powder containing approximately 4 g tuna oil was heated to 70° C. under oxygen in a sealed chamber at 5 bar, and the pressure of the sealed chamber was monitored using ML Oxipres™ (Mikrolab Aarhus A/S Denmark). The Induction Period (IP), beyond which the pressure drops dramatically due to the consumption of the oxygen, was used as an indicator to assess the oxidative stability of the LCPUFA-containing microcapsule powders. Specifically, a decrease in oxygen was recorded as the Induction Period (see FIG. 3), indicative of oxidation. Due to the high viscosity of the PPH-based tuna oil-in-water emulsion, even at 50% solid content of other formulations, it could not be spray dried, and thus oxidative stability data could not be obtained.

As shown in FIG. 3, with a surface free fat content of approximately 1%, tuna oil-containing microcapsule powders produced with different encapsulants as described in Example 1, showed varying induction periods, representative of differing levels of oxidative stability:
Sodium caseinate-based powder with 28±2% oil loading—induction period of 158 h;
PPL-based powder with 48±2% oil loading—induction period of 136 h;
WPI-based powder with 28±2% oil loading—induction period of 117 h;
MRP-based powder with with 48±2% oil loading—induction period of 51 h.

Thus, at 48±2% oil loading, microencapsulated powder encapsulated using PPL exhibited significantly improved oxidative stability compared to powder encapsulated with MRPs. Without wishing to be bound by theory, the inventors suggest that during homogenisation, the proteins with smaller molecular weights in the PPL tend to more actively migrate to the oil/water interface.

A comparison between values for the induction period of PPL-and sodium caseinate-based powders, which had different tuna oil loadings (48±2% and 28±2%, respectively) needs to take into consideration the fact that 8 g PPL-based powder (containing approximately 4 g tuna oil and approximately 0.36 g vitamin C (ascorbic acid)) and 13.33 g sodium caseinate-based powder (containing approximately 4 g tuna oil and approximately 0.70 g vitamin C (sodium ascorbate, equivalent to 0.62 g ascorbic acid)) were heated at the same temperature and under the same oxygen pressure. Thus, the PPL-based powder exhibited a better oxidative stability than the sodium caseinate-based powder with less vitamin C and higher contact surface area with oxygen.

In conclusion, the inventors produced several different microencapsulation delivery systems with either 28±2% or 48±2% tuna oil loading to stabilise the tuna oil against oxidation, with a surface free fat content around 1%.
At approximately 50% oil loading, microencapsulated tuna oil-containing powder produced using PPL and carbohydrate polymers exhibited improved oxidative stability compared with microencapsulated tuna oil-containing powder produced using MRPs of sodium caseinate and carbohydrate polymers. The inventors suggest this is due to the smaller molecular weight of PPL compared with MRPs.
The PPL-based microencapsulation system, which has 48±2% tuna oil loading, provided more effective protection to the tuna oil than SC-based microencapsulation (with 28±2% tuna oil loading), even at a lower vitamin C (0.36 v.s. 0.7 g) content and higher contact surface area with oxygen (8 v.s. 13 g powder at the same temperature and under the same oxygen pressure).
By using PPL as the protein encapsulant component, microcapsule powders with approximately 50% oil loading and low surface free fat content (1%) can be produced without Maillard reaction.
PPL, which contains protease inhibitor I (molecular weight of 39 kDa), carboxypeptidase inhibitor (molecular weight of 4100 Da), protease inhibitor IIa and IIb (molecular weight of about 20.7 kDa) and protease inhibitor A5 (molecular weight of about 20.7 kDa) can be used as an exemplary low molecular weight protein component to produce a novel microencapsulation matrix for the stabilisation of bioactive omega-3 oils.

1. A microencapsulted composition comprising one or more long chain polyunsaturated fatty acids (LCPUFAs), wherein the encapsulant comprises proteins, wherein said proteins consist of one or more low molecular weight proteins having a molecular weight of less than about 50 kDa.

2. A microencapsulated composition according to claim 1, wherein the molecular weight of the one or more proteins is less than about 40 kDa, less than about 30 kDa, or less than about 20 kDa.

3. A microencapsulated composition according to claim 1, wherein the one or more proteins are in the form of a low molecular weight protein fraction.

4. A microencapsulated composition according to claim 1, wherein the encapsulant further comprises one or more carbohydrates.

5. A microencapsulated composition according to claim 4, wherein the one or more carbohydrates are selected from maltodextrin and dextrose monohydrate, or a combination thereof.

6. A microencapsulated composition according to claim 1, wherein the one or more proteins are present at from about 5% w/w to about 25% w/w based on the total weight of the composition.

7. A microencapsulated composition according to claim 1, wherein the ratio of the protein component of the encapsulant to the carbohydrate component of the encapsulant is in the range of about 1:10 to 1:2.

8. A microencapsulated composition according to claim 1, wherein the LCPUFAs are omega-3 fatty acids.

9. A microencapsulated composition according to claim 1, wherein the LCPUFAs are present in triglyceride form.

10. A microencapsulated composition according to claim 1, wherein the LCPUFAs are present in one or more LCPUFA-containing oils.

11. A microencapsulated composition according to claim 10, wherein the one or more oils comprise a fish oil.

12. A microencapsulated composition according to claim 1, wherein the composition further comprises at least one source of vitamin C.

13. A microencapsulated composition according to claim 1, wherein the composition has a surface free fat content of about 1%.

14. A microencapsulated composition according to claim 1, wherein the composition is in the form of an oil-in-water emulsion.

15. A microencapsulated composition according to claim 1, wherein the composition is in the form of a spray dried powder.

16. A microencapsulated composition according to claim 1, wherein the one or more LCPUFAs are in triglyceride form, and wherein the composition has a surface free fat content of about 1%.

17. A method for protecting one or more LCPUFAs, or one or more oils comprising the one or more LCPUFAs, from oxidative degradation, comprising encapsulating the LCPUFAs or oil with an encapsulant comprising proteins, wherein said proteins consist of one or more low molecular weight proteins having a molecular weight of less than about 50 kDa, thereby improving the oxidative stability of the one or more LCPUFAs or oils.

19. A method according to claim 17, wherein the one or more LCPUFAs are in triglyceride form.

20. A stable emulsion comprising one or more LCPUFAs or one or more oils comprising one or more LCPUFAs, wherein said emulsion further comprises one or more proteins, wherein said proteins consist of low molecular weight proteins having a molecular weight of less than about 50 kDa.

21. A stable emulsion according to claim 20, wherein the one or more LCPUFAs are in triglyceride form.
